(19) United States
(12) Patent Application Publication
(10) Pub. No.: US 2010/0168642 A1
(43) Pub. Date: Jul. 1, 2010

(54) Multilumen Venous Catheter and Method of Use

(75) Inventors: William M. Appling, Granville, NY (US); Bettina N. Barth-King, Hague, NY (US)

Correspondence Address: ANGLODYNAMICS, INC., 14 PLAZA DRIVE, LATHAM, NY, 12110, US

(73) Assignee: ANGIODYNAMICS, INC., Queensbury, NY (US)

(21) Appl. No.: 12/648,169

(22) Filed: Dec. 28, 2009

Related U.S. Application Data
(60) Provisional application No. 61/141,513, filed on Dec. 30, 2008.

(51) Int. Cl. A61M 25/00; B23P 6/00

(52) U.S. Cl. 604/6.16; 29/402.09

(57) ABSTRACT

A multilumen venous access catheter and method of use and repair is provided. The catheter has a means for identifying at least one physical characteristic of an arterial tube and a means for identifying at least one physical characteristic of a venous tube. The at least one physical characteristic of the respective arterial and venous tubes are different in order to allow a user to distinguish between the arterial and venous catheter tubes during use, manufacture, and repair.

### CROSS-REFERENCE TO RELATED APPLICATIONS

This application claims priority to U.S. Application Ser. No. 61/141,513, filed Dec. 30, 2008. This application is also related to commonly owned U.S. patent application Ser. No. 12/648,153, filed concurrently herewith, which is incorporated herein by reference in its entirety.

### TECHNICAL FIELD

The present invention relates to a medical device and method, and more particularly, a multilumen dialysis catheter and method of use.

### BACKGROUND OF THE INVENTION

Vascular access catheters provide venous access to the central circulatory system of a patient. One example of a venous catheter is a hemodialysis catheter which provides access to the venous system during hemodialysis, a medical procedure used to cleanse the blood of patients whose kidneys do not function properly. Dialysis catheters are typically tunneled through the chest wall tissue, entering the internal jugular vein at the neck area. Alternatively, they can also be placed in the subclavian vein just below the collar bone. With dialysis catheters, it is crucial to have high flow rates in order to provide faster and more effective blood cleansing. Optimal location of the catheter tip in a large blood vessel is also necessary in order to decrease the rate of catheter occlusion.

Dual lumen dialysis catheters have been designed using two tubes with longitudinal lumens extending the length of the catheter. One lumen is dedicated for withdrawal of blood to be cleansed, known as the arterial or aspiration lumen, and another lumen is dedicated for return of the cleansed blood to the central circulatory system. This lumen is also known as the venous or return lumen. Multilumen catheters as described above are typically constructed of a single thermoplastic material such as polyurethane. Both catheter tubes can be extruded and then bonded together to form a unitary catheter shaft for at least a portion of the overall catheter length.

Although each tube of a multilumen catheter is typically formed of the same material and shape, the arterial and venous tubes are subjected to different clinical conditions. For example, the venous lumen tube is subjected to positive pressure as blood is returned to the patient. Typically pressures may reach 200 mm Hg. The arterial lumen is subject to negative pressures as the blood is pulled from the patient through the arterial lumen. These negative pressures may reach over 120 mm Hg. One problem with the use of a single material and a single lumen configuration to form both catheter tubes is that the characteristics of each catheter tube cannot be optimized to accommodate the different clinical requirements for the arterial and venous functions. Catheter tubes which are typically made of the same color can make differentiating the arterial and venous tubes of traditional dialysis catheters difficult for a practitioner during use since the distal portions of both tubes are located within the vessel and are not visible. Accordingly, it is possible for the user to unintentionally connect the arterial tube to the venous or return port on the dialysis machine. When this occurs, the dialysis procedure can be compromised due to the increased probability of blood recirculation.

To avoid this problem, extension tubes of chronic dialysis catheters are sometimes color coded, or they may include proximal luer connectors that are color coded to identify the venous from the arterial fluid channels. The extension tubes may become damaged or otherwise compromised after extended uses. When this occurs, it is preferable to repair the catheter rather than exchange the catheter, to avoid increased risk of infection to the patient. To repair the catheter, the extension tubes are cut proximal to the bifurcate hub and replaced with new extension tubes. A compression fitting is typically used to ensure an adequate seal between the original extension hub and the new extension tube section. When the damaged extension tubes are cut, the standard color coded luer connectors are also removed. Once the luer connectors have been removed, the practitioner has no way of identifying which fluid channel is associated with the arterial lumen versus the venous lumen, and may connect the repair extension tubes to the wrong lumen.

Being able to quickly and accurately differentiate the venous versus the arterial tube segments is also important during the catheter manufacturing process. Typically, both the arterial and venous catheter shafts are made of identical materials and color. The only distinguishing feature is the relative lengths of the shafts. During assembly, manufacturing personnel must assemble the correct colored luer and extension tube to the correct catheter shaft tube of the appropriate length. Because both the arterial and venous catheter tubes 5, 7 are made of the same material and color, this process can be prone to mistakes.

A catheter has not been proposed that addresses the above-mentioned problems. Thus, there is a need for a multilumen dialysis catheter comprised of at least an arterial and a venous tube, with each tube having different physical characteristics to optimize performance, integrity and longevity of the catheter, and identification of the arterial and venous lumens.

It is a purpose of this invention to provide a multilumen catheter having at least a first catheter tube and a second catheter tube, each having a lumen, in which one tube can have at least a first characteristic, and a second tube can have at least a second characteristic in order to more easily identify the first catheter tube from the second catheter tube.

It is a further purpose of this invention to provide a multilumen catheter having two different tubes that may contain different polymer materials with different durometers.

It is another purpose of this invention to provide a multilumen catheter having two different tubes that may exhibit different tensile strengths and flexibility.

It is a further purpose of this invention to provide a multilumen catheter having a first catheter tube that has a higher percentage by weight of radiopaque filler material than the percentage by weight of the radiopaque filler material contained in a second catheter tube.

It is a further purpose of this invention to provide a multilumen catheter having a first tube that has a color or pattern that may be of a different color or pattern compared to a second tube, such that the tubes are distinguishable from each other.

It is a further purpose of this invention to provide a multilumen catheter having a first arterial tube having a transverse cross-sectional profile that is different than the transverse cross-sectional profile of a second venous catheter tube to prevent the collapse of the first tube during operation and to optimize the functioning of the venous tube during operation.

Various other aspects and embodiments of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention may be found in the Detailed Description.

### SUMMARY

A multilumen catheter is provided that has a first catheter tube having at least one physical characteristic and a second catheter tube having at least one physical characteristic. The at least one physical characteristic of the respective first and second tubes are different.

A method for using a vascular access catheter is also provided herein. The method involves providing a catheter having an arterial tube and a venous tube, each tube having a means for identifying at least one physical characteristic of the arterial tube and the venous tube. At least one physical characteristic of the respective arterial and venous tubes are different. The method also involves perceiving the means for identifying at least one characteristic of at least one of the arterial tube and the venous tube; and identifying the at least one characteristic of at least one of the arterial tube and the venous tube in response to perceiving the means for identifying at least one characteristic of at least one of the arterial tube and the venous tube.

A method of repairing a catheter is also provided herein. This method involves providing a catheter having at least one extension tube; cutting at least a portion of at least one extension tube of the catheter; and providing a repair kit. The repair kit has at least one replacement extension tube, and at least a portion of the replacement extension tube is identical in at least one visible color to at least a portion of at least one extension tube of the catheter. The repair kit also has at least one means for sealing at least a portion of the replacement extension tube to at least a portion of the extension tube of the catheter. The method further involves attaching at least a portion of the sealing means to at least a portion of the extension tube of the catheter by matching the at least one visible color of the replacement extension tube with an identical visible color of at least a portion of the extension tube of the catheter and securing at least a portion of the extension tube of the catheter to at least a portion of the sealing means of the repair kit.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 illustrates a plan view of one embodiment of a multilumen hemodialysis catheter assembly.

FIGS. 2A through 2C illustrate cross-sectional views of the catheter shaft of the catheter assembly of FIG. 1 taken along lines 2A-2A, 2B-2B, and 2C-2C of the catheter shaft of FIG. 1.

FIGS. 3A and 3B illustrate cross-sectional views of an alternative embodiment of the catheter shaft along line 2A-2A of FIG. 1.

FIG. 4 illustrates the catheter assembly of FIG. 1 after the removal of the extension tubes and prior to re-assembly with new extension tube sets.

FIG. 5 illustrates another embodiment of the catheter assembly with a different distal tip configuration.

### DETAILED DESCRIPTION OF THE INVENTION

The present invention can be understood more readily by reference to the following detailed description and the examples included therein and to the Figures and their previous and following description. The drawings, which are not necessarily to scale, depict selected preferred embodiments and are not intended to limit the scope of the invention. The detailed description illustrates by way of example, not by way of limitation, the principles of the invention.

The skilled artisan will readily appreciate that the devices and methods described herein are merely exemplary and that variations can be made without departing from the spirit and scope of the invention. It is also to be understood that the terminology used herein is for the purpose of describing particular embodiments only and is not intended to be limiting.

Ranges can be expressed herein as from “about” to one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. As used herein, the words “proximal” and “distal” refer to directions away from and closer to, respectively, the insertion tip of the catheter in the catheter assembly. The terminology includes the words above specifically mentioned, derivatives thereof, and words of similar import.

“Optional” or “optionally” means that the subsequently described element, event or circumstance can or cannot occur, and that the description includes instances where said element, event or circumstance occurs and instances where it does not.

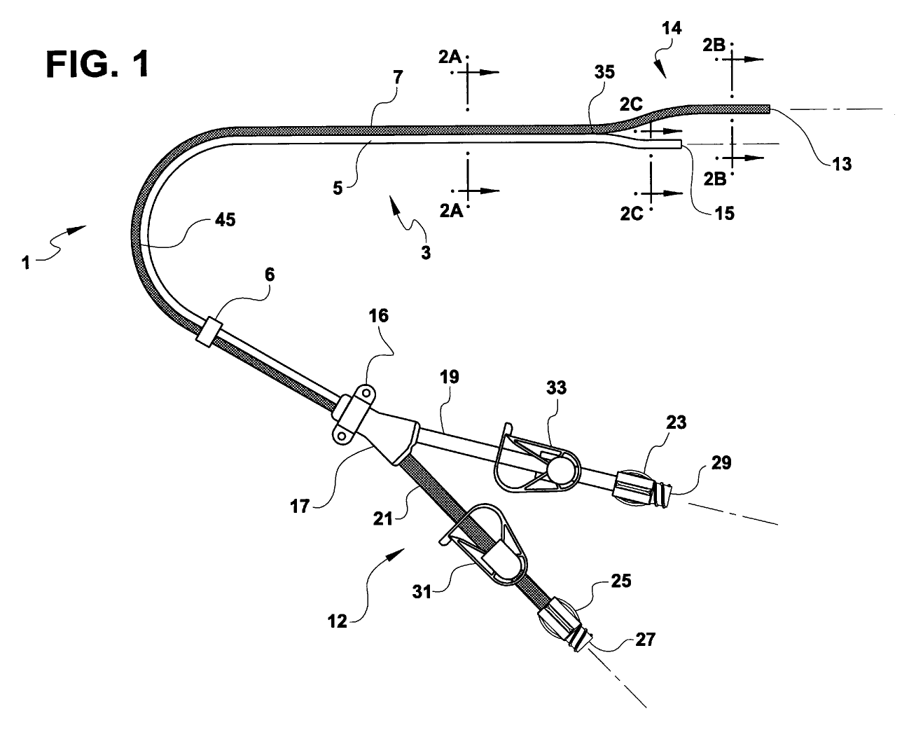

Referring now in detail to the drawings, in which like reference numerals indicate like parts or elements throughout the several views, in various embodiments, and referring to FIGS. 1-5, presented herein is an exemplary multilumen vascular access catheter, such as hemodialysis catheter and a method of using the catheter that involves identifying and distinguishing at least one catheter tube from another catheter tube. Also presented herein is a repair kit for repairing a catheter and a method of repairing a catheter using the catheter repair kit. In one aspect, although the catheter configuration described herein is for a dialysis catheter, this catheter configuration can be used to produce any type of multilumen catheter, such as, but not limited to, peripherally inserted central catheters (PICCs), angiographic catheters, or other types of catheters.

FIG. 1 illustrates one exemplary embodiment of a multilumen vascular access catheter assembly 1, such as a hemodialysis catheter. The catheter assembly 1 has a proximal end 12 that is adapted to remain outside of a patient's body and a distal end 14 that is adapted for insertion into a patient's blood vessel, such as, but not limited to, a vein. In one aspect, the catheter assembly 1 is comprised of an elongate unitary catheter shaft or tube 3. In one aspect, at least a portion of the catheter shaft 3 forms a distal portion 14 of the catheter. In one aspect, the distal portion 14 of the elongate catheter shaft 3 comprises separable distal tip portions that are adapted for insertion into a blood vessel.

In one aspect, the catheter shaft 3 can be comprised of a plurality of tubes. In one aspect, the catheter shaft 3 can be comprised of at least a first tube 5 and a second tube 7. In one aspect, the tubes 5, 7 can extend substantially the entire length of the catheter shaft 3. In another exemplary aspect, one or both of the tubes 5, 7 can extend a partial length of the catheter shaft 3. In one aspect, each catheter tube 5, 7 of catheter shaft 3 can have at least one unique physical characteristic that can be customized based on the intended function of each tube 5, 7, as represented by the different shadings of catheter tubes 5, 7. In one aspect, tube 5 can have a first characteristic, and tube 7 can have a second characteristic. Each of the first characteristic and the second characteristic can correspond to at least one visible color. The first characteristic of the first tube can be different from the second characteristic of the second tube. More particularly, at least a portion of the first tube can differ in at least one visible color from at least a portion of the second tube. At least one characteristic of each tube 5, 7 can be identifiable according to the at least one visible color. For instance, at least a portion of the first tube can differ in at least one visible color from at least a portion of the second tube, and at least one characteristic of each tube is identifiable according to the at least one visible color. Physical characteristics may include, but are not limited to, hardness, tensile strength, durometer, rigidity, flexibility, radiopacity, cross-sectional luminal area, torquability, trackability, pushability, surface finish, materials, fatigue strength, percentage of radiopaque filler material, abrasion resistance, color, arterial tube function, venous tube function, and physical configuration.

In another aspect, other physical properties of the catheter tubes 5, 7 can be modified to optimize the overall clinical performance of the catheter. In addition to hardness, tensile strength and radiopacity, other properties may be adjusted to achieve the desired degree of abrasion resistance, fatigue strength, and long-term dimensional stability.

In one aspect, a bifurcate or hub 17, can surround at least a portion of the outer surface of the proximal portion 12 of the catheter shaft 3. In one exemplary aspect, the bifurcate 17 can be composed of Carbothane® PC3585A-B20. Cuff 6, which facilitates anchoring for tunneled catheters, may optionally be attached to at least a portion of at least one of the outer surfaces of catheter tubes 5, 7 of the unitary catheter shaft 3. In one aspect, the cuff 6 can be useful for allowing subcutaneous tissue to grow into the cuff and to help secure the catheter once it is implanted in a patient's body. In one exemplary aspect, the cuff 6 can be comprised of polyester or Dacron.

In one aspect, the catheter assembly 1 can have at least a first extension tube 19 and a second extension tube 21. In one aspect, the catheter assembly 1 can have at least a first extension tube clamp 33 and a second extension tube clamp 31. In one aspect, the clamps 33, 31 can be releasably attached to at least a portion of each extension tube 19, 21, respectively. In one aspect, the clamps 31, 33 can be releasably attached to at least a portion of the outer surface of at least one of the extension tubes 19, 21. In one aspect, optionally at least one of the catheter extension tubes 19, 21 can have at least one pre-curved portion. The at least one pre-curved portion can enable at least one of the extension tubes 19, 21 to extend downward against a patient's body once the distal portion 14 of the catheter assembly 1 has been placed in a patient's vasculature. This design is beneficial because it can provide greater comfort for the patient. In one exemplary aspect, the extension tubes 19, 21 can be made of clear Carbothane® PC3595A. In another exemplary aspect, the clamps 31, 33 can be composed of Acetal/Tecoflex.

In one aspect, the catheter assembly 1 has at least a first catheter hub connector or luer connector 23 and a second catheter hub connector or luer connector 25 for joining to a dialysis machine or other injection or aspiration device in order to provide intravascular access to a patient. In one exemplary aspect, the luer connectors 23, 25 can be composed of Isoplast 2510. In another aspect, the catheter shaft 3 can have at least one suture wing 16 for providing securement of the catheter body to the patient. In one exemplary aspect, the suture wing 16 can be composed of Pellathane®.

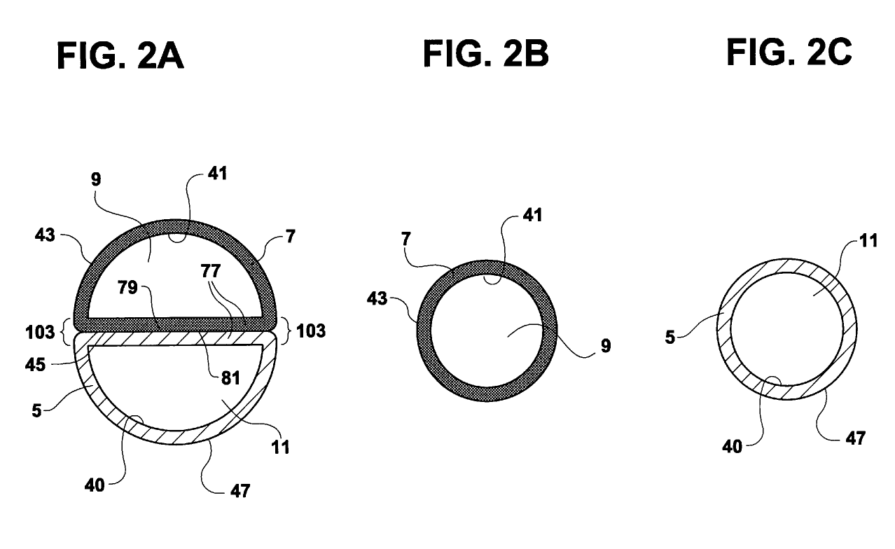

In one aspect, the plurality of tubes of the catheter shaft 3 can comprise a plurality of lumens 9, 11 (FIG. 2A) that can extend along a longitudinal axis for at least a portion of the catheter shaft 3. In one aspect, the lumens 9, 11 can extend from the proximal end 12 of the catheter shaft 3 to the distal end 14 of the catheter shaft 3. In one aspect, each of the catheter lumens 9, 11 can comprise at least one aperture. More particularly, in one aspect, each of the lumens 9, 11 can comprise at least a proximal aperture and a distal aperture 13, 15, respectively. Aperture 13 of first lumen 9 can be defined in at least a portion of a distal end portion of the catheter shaft 3. Aperture 15 of second lumen 11 can be defined in at least a portion of a distal end portion of the catheter shaft 3. In one aspect, distal apertures 13, 15 can be defined in a distal most portion of each of the catheter tubes 7, 5, respectively. In one aspect, each of the lumens 9, 11 of the catheter tubes 5, 7 terminates in a distal aperture 13, 15, respectively. In one aspect, each of the distal apertures 13, 15 can be in fluid communication with at least a portion of the interior of the catheter shaft 3. More particularly, in one aspect, each of the distal apertures 13, 15 can be in fluid communication with at least a portion of lumens 9, 11, respectively. In one aspect, catheter tubes 5, 7 can have at least one aperture that can be defined within a portion of the sidewall of the catheter tubes 5, 7 anywhere along the length of the catheter shaft 3 such that the at least one sidewall aperture can be in fluid communication with at least a portion of the catheter lumens 9, 11.

When the catheter assembly 1 is connected to a dialysis machine, blood is withdrawn from a patient's venous system and transported through second withdrawal lumen or arterial lumen 11, illustrated in FIG. 2A, of withdrawal or aspiration tube 5 at aperture 15 for cleansing by the dialysis machine. Aspiration of the blood is accomplished by drawing a vacuum or negative pressure through opening 29 of luer connector 23, causing the blood to be drawn through the catheter into the dialysis machine. The treated blood is then returned to the central venous system through opening 27 of luer connector 25 which is connected to the dialysis machine, into infusion or venous tube 7 with first lumen or venous lumen 9, under pressure, through distal aperture 13 into the patient's bloodstream. In one aspect, extension tubes 19, 21 are fluidly joined with lumens 9, 11, respectively, so as to enable the infusion or aspiration of fluids from or to the central venous system of a patient.

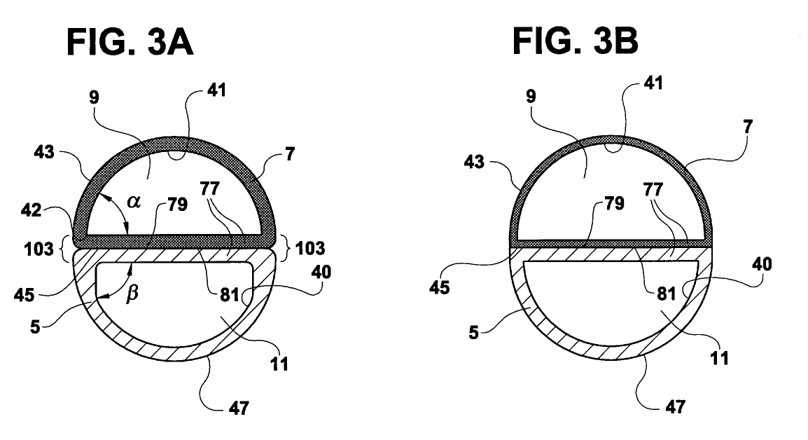

As illustrated in FIGS. 1, 2A, 3A, 3B, 4, and 5, at least a portion of each of catheter tubes 5, 7 are bonded or fused to one another, without the use of an adhesive, along a joining line or interface 45 to form a catheter shaft 3 having a unitary continuous smooth exterior or outer surface for at least a portion of the overall catheter length from the hub 17 to a pre-determined dividing point 35. More particularly, in one aspect, at least a portion of the catheter tubes 5, 7 can be joined or fused together along at least a portion of the catheter length using the method described in U.S. application Ser. No. 12/648,153, filed concurrently herewith, which is incorporated herein by reference. In one aspect, at least a portion of the surfaces of tubes 5, 7 can be joined together by using at least a portion of a heating means such as, but not limited to, a heating block. More particularly, in one aspect, at least a portion of the inner surfaces 79, 81 of the at least two catheter tubes 5, 7 can be heated using the heating means. In one aspect, at least a portion of the inner surfaces 79, 81 of the catheter tubes 5, 7 can be uniformly heated as they are pulled over a portion of the heating means surface. At least a portion of the catheter 5, 7 surfaces can have substantially equal contact with at least a portion of the heating means surface. In one aspect, after at least a portion of the inner opposed surfaces 79, 81 of each of the catheter tubes 5, 7 are heated across a portion of the heating means surface, such that at least a portion of each of the catheter tube 5, 7 surfaces can heat bond or fuse together, thereby forming a unitary septum 77, as illustrated in FIGS. 2A, 3A, and 3B. In one aspect, the inner opposing surfaces 79, 81 can be substantially flat or planar.

Alternatively, in one aspect, heat can be applied to at least a portion of the inner surfaces 79, 81 of each catheter tube 5, 7, such that only a portion of the inner surfaces 79, 81 are heated, thereby creating a third lumen between the two catheter tubes 5, 7, as illustrated in U.S. application Ser. No. 12/648,153, filed concurrently herewith. Thus, the method used to manufacture the multilumen catheter described herein can comprise providing a plurality of tubes, each tube having a first end, a second end, at least one lumen extending longitudinally through at least a portion of each tube, and at least one surface; selectively heating at least a portion of the at least one surface of at least a first tube; and contacting the selectively heated portions of the first tube with the second tube to form a multilumen catheter shaft that is joined together along at least a portion of the length of the catheter shaft. Alternatively, a portion of the surface of the second catheter tube can be selectively heated also, and the selectively heated portions of the first tube and the second tube can be joined together to form a unitary catheter shaft.

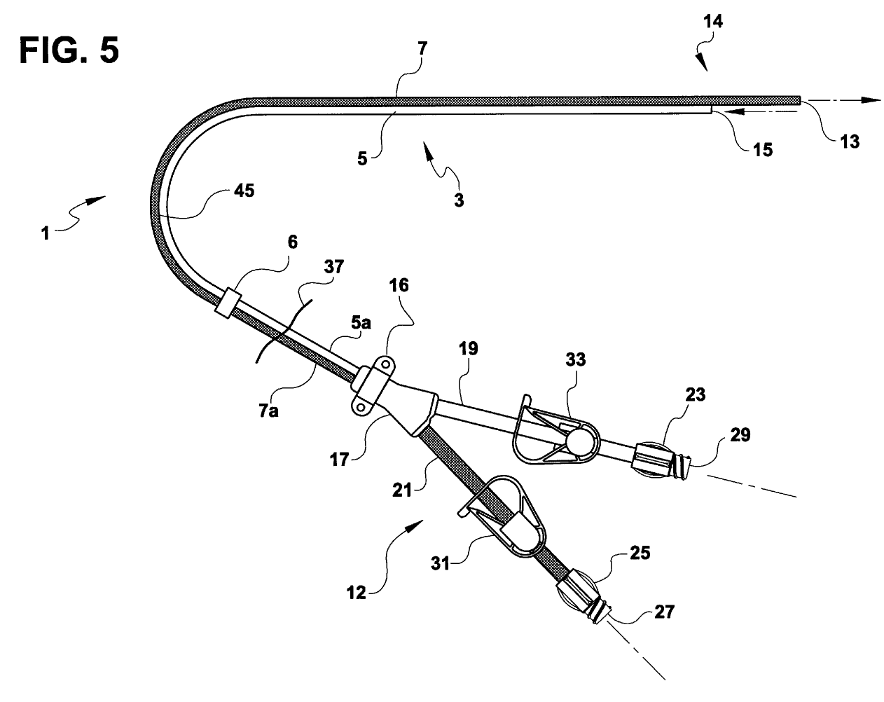

In one aspect the multilumen catheter shaft 3 described herein can have various types of distal tip configurations, such as, but not limited to distal tip portions where the distal tips are the same length, different lengths (FIG. 1), straight (FIG. 5), or curved (FIG. 1). In one aspect, the catheter tube configuration described herein may be used with other types of catheter configurations, such as, but not limited to, split tip, staggered (FIG. 5), or other distal tip catheter configurations. In one aspect, catheter tubes 5, 7 can be of different lengths help to prevent recirculation of non-purified blood with purified blood. As illustrated in FIGS. 1, 4, and 5, in one aspect, the first, venous lumen 9 can be longer in length than the second, arterial lumen 11. The shorter arterial lumen and the longer venous lumen are critical for preventing recirculation. Apertures 13, 15 of the catheter assembly 1 can be spaced from each other in order to minimize recirculation of the returned blood into the inlet aperture 15. This spacing is critical to prevent recirculation.

In one aspect, “split tip” catheter is defined herein to mean the catheter assembly 1 described herein having a catheter shaft 3 comprising at least two lumens 9, 11 and a separation point 35 that separates at least two distal tip portions. In one aspect, the separation point 35 can be located anywhere along the length of the catheter shaft 3. Each of the distal tip portions can enclose at least one lumen 9, 11 and are separable from one another along their length. In one aspect, the distal end portions of the distal portion 14 of the catheter tubes 5, 7 can have a pre-shaped memory so that they assume a predetermined shape when not under the influence of an external force.

In one aspect, at least a portion of the surfaces of each of the catheter tubes 5, 7 can be permanently joined together for at least a partial length of the catheter shaft 3. Alternatively, the tubes 5, 7 can be splittable or releasably attached. Splittable or releasably attached means that at least a portion of the distal end portions of the catheter tubes 5, 7 of catheter shaft 3 are capable of becoming separated or unattached from the distal end portions proximal to dividing point 35 upon applying minimal force to at least a portion of the catheter tubes 5, 7. In one aspect, approximately one to five pounds of force can be sufficient to separate at least a portion of the catheter tubes 5, 7. After at least a portion of the tubes 5, 7 are pulled apart, the tubes 5, 7 can become free-floating relative to each other. In one aspect, at least a portion of the catheter tubes 5, 7 can be longitudinally split apart along a longitudinal axis by holding at least a portion of each tube 5, 7 and manually pulling each tube 5, 7 in an opposite direction from each other distally of dividing point 35. This allows the catheter tubes 5, 7 to be capable of independent movement relative to one another such that they are not attached to each other distally of dividing point 35. The releasably attached or splittable catheter shaft 3 allows for adaptability and flexibility of use and insertion of the catheter assembly 1. In one aspect, the length of the distal portion of the catheter tubes 5, 7 can be adjusted to accommodate patients of various heights and weights.

As illustrated in FIGS. 2A through 2C, several cross sectional views of the distal portion 14 of the catheter assembly of FIG. 1 are shown. In one aspect, lumens 9, 11 can extend generally longitudinally parallel to each other along a longitudinal axis, such that lumen 9 is positioned on a first side of the longitudinal axis, and lumen 11 is positioned on a second side of the longitudinal axis. As shown along the cross-section 2A-2A of FIG. 1, in one aspect, tube 5 has an outer surface 47 and tube 7 has an outer surface 43. Arterial tube 5 or first tube 5 has an arterial lumen 11 with an inner lumen surface 40. In one aspect, the portion of the catheter tube 5 that is defined between the outer surface 47 and the inner lumen surface 40 defines the tube 5 sidewall. In one aspect, tube 5 can have an outer surface 47 and an inner surface 81. In one aspect, second tube or venous tube 7 has a venous lumen 9 with an inner lumen surface 41 and an outer surface 43. Tube 7 can have an outer surface 43 and an inner surface 79. In one aspect, the portion of the catheter tube 7 that is defined between the outer surface 43 and the inner lumen surface 41 defines the tube 7 sidewall.

At least a portion of the catheter tubes 5, 7 can be joined together along at least a portion of inner surfaces 79, 81 such that the combined surfaces form an integral, internal, bisecting planar septum 77, illustrated in FIGS. 2A, 3A, and 3B. In one aspect, the septum 77 extends diametrically across the interior of the catheter shaft 3 and defines lumens 9, 11 such that both lumens 9, 11 are substantially D-shaped in transverse cross-section. In one aspect, the septum 77 has a uniform thickness. Although shown in a round transverse cross-sectional shape along the distal portion 14 of the catheter assembly 1, as illustrated in FIGS. 2B and 2C, the tubes 5, 7 may be of other cross-sectional profiles including oval, square, triangular, kidney bean, elliptical, or any other suitable lumen configuration, diameter, material, thickness, or length, in any combination thereof along the catheter shaft 3. Cross-sectional lumen shapes may be uniquely extruded to optimize flow and pressure requirements. For example, the traditional venous D-shaped lumen, designed to maximize flow rates, may be modified for the arterial tube 5 to reduce the possibility of arterial lumen collapse. In one aspect, the surface finish of the inner walls 40, 41 of the arterial and venous lumens, respectively, can also be modified to optimize both negative and positive pressure flows.

In one exemplary aspect, as illustrated in FIG. 2A, a slight indentation or “v-shape” 103 can be defined in the outer surface of the catheter shaft 3 as a result of the manufacturing process described in U.S. application Ser. No. 12/648,153, incorporated herein by reference. This “v-shape” can be defined at opposing outer edges of the catheter tubes 5, 7, as illustrated in FIG. 2A, when at least a portion of the catheter tubes 5, 7 have been joined together. The v-shaped indentation 103 can be varied such that it is clearly visible to an observer. In another exemplary embodiment, the v-shape can be visible only using microscopic guidance. In one aspect, this slight v-shaped indentation 103 can serve to assist the user or manufacturer in identifying the location and type of the at least two catheter tubes 5, 7 relative to each other. In yet another exemplary embodiment, as illustrated in FIG. 3B, the outer surface of the catheter shaft 3 can be uniformly smooth across substantially the entire outer surface of the catheter shaft 3 such that no v-shape indentation is visible in the outer surface of the catheter shaft 3 either to an ordinary observer or under microscopic guidance. The physical characteristics of catheter tubes 5 and 7 may be selected so as to optimize the functioning of the catheter during insertion, use and repair of the catheter. Each tube can exhibit unique physical characteristics optimized for either venous or arterial flow. Each tube can also be uniquely designed to minimize complications related to placement and catheter dysfunction after placement. Characteristics include but are not limited to rigidity, tensile strength, cross-sectional lumen shapes, radiopacity, surface finish, and color. Material selection, percentage of filler, color, and lumen dimensions may be varied to achieve tubing specifications unique to each tube of the catheter.

In one aspect, the catheter assembly 1 can comprise a means for identifying at least one physical characteristic of at least one of the tubes 5, 7. In one aspect, the means for identifying at least one physical characteristic can be used to identify at least one physical characteristic of the arterial tube that is used to prevent the collapse of the arterial tube 5 during use. In another aspect, the catheter assembly 1 can comprise a means for identifying at least one physical characteristic of a venous tube 7 to optimize the functioning of the venous tube 7 during use. At least one of the characteristics of the venous tube 7 and the arterial tube 5 can be different from each other. In one aspect, the means for identifying at least one physical characteristic can vary. In one exemplary embodiment, the means can comprise, but is not limited to, the catheter tube wall thickness, at least one visible color of the catheter tube, the durometer of the catheter tubes, radiopaque material embedded within on or at least a portion of at least one catheter tubes, or any other patterns or textures of the catheter tubes 5, 7 and the like.

In one aspect, catheter tubes 5, 7 are preferably designed to maximize the cross-sectional diameter of the lumens 9, 11 to achieve increased flow rates during dialysis. As illustrated in FIG. 2A, in one aspect, the transverse cross-sectional area of the substantially D-shaped lumens 9, 11 can be substantially equal. In one aspect, lumen 11 can have a first transverse cross-sectional area, and lumen 9 can have a second transverse cross-sectional area. In one aspect, the first transverse cross-sectional area can be smaller than or larger than the second transverse cross-sectional area along at least a portion of the longitudinal length of the catheter shaft 3, depending on the desired flow rates. In one aspect, the venous and arterial lumens 9, 11 can be configured so as to accommodate fluid flow rates required for hemodialysis, i.e., about 300 ml/min. at about 250 mm Hg pressure. In one aspect, both lumens 9, 11 are configured to remain patent during use such that they do not collapse. The overall cross-sectional area of the lumens 9, 11 remains substantially equal or constant during use. This helps to ensure that the overall cross-sectional area of the catheter is not compromised during use and that adequate flow rates can be maintained. Thus, the overall cross-sectional area of the catheter remains substantially constant before, during, and after use. Although primarily used for hemodialysis, the multilumen catheter described herein can also be used for other exemplary processes, such as, but not limited to, plasmapheresis, perfusion, infusion, and chemotherapy.

In one aspect, the venous tube 7 can be used to return cleansed blood back into the central circulatory system. Positive pressure generated by the dialysis pump pushes the blood through the lumen 11 and into the patient. The positive pressure used to generate forward movement of the blood makes the venous tube 7 less likely to collapse during use. As a result, the venous tube 7 is less susceptible to lumen collapse than an arterial tube with the same physical characteristics. To accommodate for this difference, in one exemplary embodiment, the arterial tube 5 may be constructed of at least one type of material having characteristics that provide increased rigidity to ensure that the lumen does not collapse when exposed to negative aspiration pressures. Thus, the arterial tube 5 of the multilumen venous catheter can have at least one physical characteristic that can allow the arterial tube to be configured to prevent lumen collapse under negative pressure. The arterial tube 5 may be comprised of a polymer material designed to withstand dialysis treatment conditions without collapse. In one aspect, the arterial tube 5 may be comprised of materials such as, but not limited to, polymers such as polyesters, polyurethanes, polyamides, polyesters, polyolefins such as polypropylene and polyethylene, and other thermoplastic polymers. In one aspect, it is contemplated that the venous and arterial tubes may be comprised of different materials, such that the materials of the venous and arterial tubes differ enough chemically so as to enable a user to distinguish the respective tubes from one another. In one aspect, the venous tube 7 may be constructed of at least one material exhibiting a lower tensile strength compared to the material used for the arterial tube 5 having more flexibility to minimize trauma to the vessel wall.

In one aspect, the polymer material of the arterial tube may be selected so as to have a higher radial and tensile strength than a venous tube 7 having the same dimensions. The durometer of the polymer material used in the tubes 5, 7, measured in shore A hardness, may, for example, be about 55D shore hardness for the arterial tube 5 and about 75A shore hardness for the venous tube 7. The higher durometer polymer (55D) provides increased tensile strength and durability so as to minimize the possibility of lumen collapse during the application of negative pressure to the tube 5. Alternatively, the durometer of the venous tube can be greater than the durometer of the arterial tube.

Alternatively, or in addition to, the polymer material may be combined with a radiopaque filler material to customize the physical characteristics of the venous and arterial tubes 5, 7. Radiopaque filler material can be used to enhance the visibility of a device under fluoroscopy. Placement of the catheter 1 with the venous tube 7 distal tip aperture 13 in a large blood vessel such as the right atrium is critical to achieving optimal flow rates. Positioning the venous catheter distal tip aperture 13 within the right atrium provides high blood flow rates required for efficient dialysis and thus more effective treatment. Optimal location of the catheter distal tip aperture 13 in a large blood vessel is also necessary in order to decrease the rate of catheter occlusion, which occurs when the tip 13 comes into contact or rests up against the blood vessel wall for extended periods of time. Damage to the vessel wall may lead to thrombosis build up and catheter malfunction.

Radiopaque loading percentages in the catheter tube sidewalls of the arterial and venous tubes may also be varied based on the tube function. In one exemplary aspect of the present invention, the venous tube 7 may have an increased level of radiopaque filler material relative to the arterial tube 5. Increased radiopacity allows a practitioner to more accurately position the venous tip or aperture 13 within the right atrium due to the increased visibility of the tip 13 under fluoroscopy. Because the radiopaque-loaded venous tube 7 is longer, the entire catheter length can be visible under imaging. In one exemplary aspect, only the venous tube 7 can be loaded with radiopaque filler. This helps to prevent the tensile strength and rigidity of arterial tube 5, which are required due to the negative pressure conditions during dialysis, from being compromised. Thus, the distal tip portion of the venous tube 7, which is typically longer than the arterial tube 5 to minimize recirculation, may be comprised of a higher radiopaque loading, compared to the distal tip portion of the arterial tube 5, for increased visibility of the distal most section of the catheter during placement. Conversely, the arterial tube 5 may have a lower radiopaque filler to plastic ratio to ensure that tensile strength is maintained at desired levels. Alternatively, the arterial tube can have a higher percentage by weight of radiopaque filler material.

In one exemplary aspect, radiopaque material can be embedded onto or interspersed throughout at least a portion of the inner surfaces 79, 81 of the catheter tubes 5, 7 at a pre-determined distance to create equally spaced apart radiopaque markers. Embedding radiopaque material between at least a portion of the inner surfaces 79, 81 of the catheter tubes 5, 7 can eliminate the need to put radiopaque marker bands around the outer surface of the catheter shaft. The radiopaque markers can be beneficial because they can function as an internal marker band that is embedded within the catheter shaft 3 and does not interfere with the insertion of the catheter shaft 3 or the interior of the catheter shaft 3. In one exemplary aspect, if the catheter shaft 3 is coated on the exterior surface, then the radiopaque markers will not interfere with the coating(s). This design is also beneficial because it allows fewer foreign materials to be exposed to the patient's body and fewer external bands around the catheter shaft, which bands could otherwise become dislodged or loose inside the patient. In one aspect, radiopaque material can be pre-embedded within a sidewall of at least one of the catheter tubes 5, 7. In one aspect, the radiopaque material or markers can comprise barium sulfate, zirconium dioxide, tantalum, tungsten, platinum, gold, silver, stainless steel, titanium, or any alloys thereof that are suitable for radiopacity. In one aspect, the radiopaque materials can also function as a reinforcing means of at least a portion of the tubes 5, 7. Alternatively, radiopaque material can be inserted between at least a portion of the catheter tube 5, 7 surfaces through a receiving means that can be positioned between the two catheter tubes 5, 7 surfaces during the heat-bonding manufacturing process described in U.S. application Ser. No. 12/648,153, incorporated herein by reference.

In yet another embodiment, although not illustrated, at least one electrical wire or a shape memory wire can be embedded within at least a portion of the sidewalls of catheter tubes 5, 7. In one aspect, the at least one wire can be embedded between more than one of the surfaces of the catheter tubes. In one aspect, the at least one wire can be formed of a material such as nitinol, stainless steel, nickel, titanium or alloys of nickel, or other types of suitable metal. In one aspect, if a nitinol wire is used, the wire can allow a user to twist or bend the finished unitary catheter shaft such that it has a desired bend or shape after the wire has been embedded within the catheter shaft 3. In one aspect the wire can be embedded in various positions within the unitary catheter shaft 3. The at least one embedded wire can be used to enhance the pushability, visibility, and strength of the catheter. In one aspect, the at least one wire can be radiopaque.

In one aspect, as described in U.S. application Ser. No. 11/074,504, and incorporated herein by reference, the catheter shaft 3 can include a plurality of wires that are electrodes that can be embedded anywhere within the catheter shaft. In one aspect, the electrodes can vary in size, shape, and length, can be positive or negative, and can be embedded in at least a part of the catheter shaft. In one aspect, the at least one wire can be round. In another aspect, the at least one wire can have a different shape, such as flat or curved. In one aspect, the at least one electrode can extend for at least a partial length within the catheter shaft and can connect each electrode to a source of electrical energy in the form of a generator. In one aspect, the at least one electrode can be comprised of any suitable electrically conductive material, including, but not limited to, stainless steel, gold, silver, nitinol, or other metals.

Referring to FIGS. 3A and 3B, two exemplary embodiments of cross-sectional views of the catheter lumens are illustrated. In this aspect, the arterial and venous lumens 9, 11 can be dimensioned to optimize performance of each catheter tube 5, 7. Typical dialysis catheters are designed to have a catheter wall that is as thin as possible, while achieving other requirements such as kink resistance, pushability, and tensile strength. During a dialysis session, the arterial tube 5 can be subjected to negative pressures as the blood is drawn into lumen 39. Negative pressures may exceed 120 mm Hg. These pressures may result in arterial lumen collapse, particularly if the tube walls are thin, and made of soft materials, so as to maximize flow rates. When the arterial lumen collapses upon itself under negative pressure, the dialysis session must be stopped, resulting in incomplete treatment or extended treatment times as well as patient discomfort. Partial lumen collapse may also result in thrombosis or clot build-up within the lumen.

FIG. 3A depicts a cross-sectional view of the unitary catheter shaft 3 taken along line 2A-2A of the catheter illustrated in FIG. 1. In one aspect, the venous tube 7 comprises a substantially D-shaped lumen 9. In one aspect, at least a portion of the surface of venous tube 7 is joined to at least a portion of the surface of arterial tube 5 along an interface 45, as described above. In one aspect, arterial tube 5 can have a variable sidewall thickness such that that the substantially D-shaped lumen 11 is slightly modified. In one exemplary aspect, the catheter sidewall can have a varying thickness around its circumference. In one exemplary aspect, the catheter sidewall can comprise a portion with a first sidewall thickness and a portion with a second sidewall thickness. In one aspect, the first sidewall thickness can be thicker than the second sidewall thickness. In one exemplary aspect, at least a portion of the thickness of the catheter tube 5 sidewall can be generally uniform. In another aspect, at least a portion of the catheter tube 5 sidewall can be gradually increasing. Thus, in one aspect, the outer diameter of catheter tube 5 is generally uniform along the length of the catheter shaft 3, while the inner diameter of lumen 11 can be variable.

The catheter tubes 5, 7 or first tube 5, second tube 7 sidewalls can be configured such that at least a portion of the sidewall of the first tube is thicker than the second tube sidewall or a portion of the second tube sidewall can be thicker than the first tube sidewall. In one aspect, the sidewall of the arterial catheter tube 5 can be thicker than the venous tube 7 sidewall which can be useful for increasing the rigidity of catheter tube 5 and can be configured to prevent arterial luminal collapse under negative aspiration pressure during use. One of ordinary skill in the art will recognize that the thickness of each of the catheter tubes 5, 7 can be modified, depending upon the desired rigidity. This variable thickness helps to minimize the likelihood that the catheter will kink when curved, bent, or under internal pressure during use. This can allow the arterial lumen 11 to handle a lower negative pressure during blood flow, which is required to provide adequate blood flow during dialysis. Thus, the venous lumen can be configured to optimize the venous tube functioning during use.

In this embodiment, the transverse cross-sectional are of lumen 11 can be less than the transverse cross-sectional are of lumen 9. In one aspect, as illustrated in FIG. 3A, at least a portion of the inner wall 40 of the arterial lumen 11 can define an angle β of between approximately 10 and 90 degrees. In one aspect, the angle β can be approximately 90 degrees. Conversely, in one aspect, at least a portion of the interior wall 41 of the venous lumen 9 can be defined by an angle of α. In one exemplary aspect, the angle α may be between approximately 10 and 80 degrees. In one aspect, the angle α may be approximately 67 degrees. The increased thickness of at least a portion of the wall of the arterial tube 5, combined with the angle β of 90 degrees, allows for an increased rigidity of the arterial lumen 11 relative to the venous lumen 9.

FIG. 3B depicts a cross-sectional view of another embodiment of the catheter 1 in which the arterial and venous lumens can be customized to optimize performance of the catheter assembly 1. In this aspect, the sidewall of the venous tube 7 of the catheter shaft 3 can be made of at least one material that will allow the venous shaft tube sidewall to be thin, without compromising wall strength. This can result in a larger luminal transverse cross-sectional area compared to the typical catheter assembly design described above. As illustrated in FIG. 3B, the thickness of the sidewall of venous tube 7 has been reduced to increase the cross-sectional area of the venous lumen 9, to allow for higher flow rates, while the arterial tube 5 sidewall thickness has remained the same.

In yet another aspect of the present invention, the polymer material of at least one tube 5, 7 can be loaded with at least one colorant filler, as indicated by the different catheter tube shadings. The colorant filler provides a visual indicia along at least a portion of the outer surface of each catheter tube 5, 7 for the user that helps the user to distinguish one tube from another during dialysis treatment and catheter manufacturing and repair procedures. In one aspect, at least a portion of venous tube 7 can be loaded with at least one colorant. Typically, tube 7 can comprise up to approximately 4% colorant by weight. In one aspect, the purpose of the colorant additive is to provide for immediate visual identification by a practitioner of the venous tube 7 versus the arterial tube 5. In one exemplary aspect, the color red can be used for the arterial tube 5, and the color blue can be used for the venous tube 7. In one aspect, it is generally known that the color red symbolizes arterial flow, and the color blue symbolizes venous flow in dialysis catheters.

In yet another aspect, a method of using the multilumen vascular access catheter assembly 1 of the current invention is provided. The method of use includes providing the catheter described herein, perceiving the means for identifying at least one characteristic of at least one of the arterial tube and the venous tube; and identifying the at least one characteristic of at least one of the arterial tube and the venous tube in response to perceiving the at least one characteristic of at least one of the arterial tube and the venous tube. In one aspect, the step of perceiving may involve visually perceiving the means for identifying at least one characteristic. The step of perceiving can further include distinguishing at least one color or pattern of a first catheter tube from at least one color or pattern of a second catheter tube in order to distinguish the tubes from each other. The step of identifying can further involve identifying the at least one characteristic of at least one of the arterial tube and the venous tube based on the at least one visible color. The step of perceiving the at least one characteristic can involve perceiving at least one of the following characteristics: hardness, durometer, rigidity, flexibility, radiopacity, torquability, trackability, pushability, fatigue strength, abrasion resistance, arterial tube function, venous tube function, and physical configuration.

Inadvertently connecting the arterial tube 5 to the venous connection on the dialysis machine (or vice versa) can result in compromised flow levels and increased recirculation rates. Therefore, it is critical for a user to be able to clearly and quickly identify which tube should be connected to which dialysis catheter connection. Although prior art catheters may have uniquely colored clamps 31 and 33 to assist in uniquely identifying each tube, these clamps are removed if the catheter must be repaired while still implanted in a patient. Once removed, there is no way for the practitioner to uniquely identify the venous versus arterial tubes. Thus, use of colors such as blue and red for the catheter tubes 5, 7, respectively, may make identification of the catheter tubes 5, 7 easier for users. It is contemplated that any suitable distinguishable color or colored pattern may be used for each catheter tube, as long as the colors or patterns are useful for visually identifying and distinguishing at least one from the other tube.

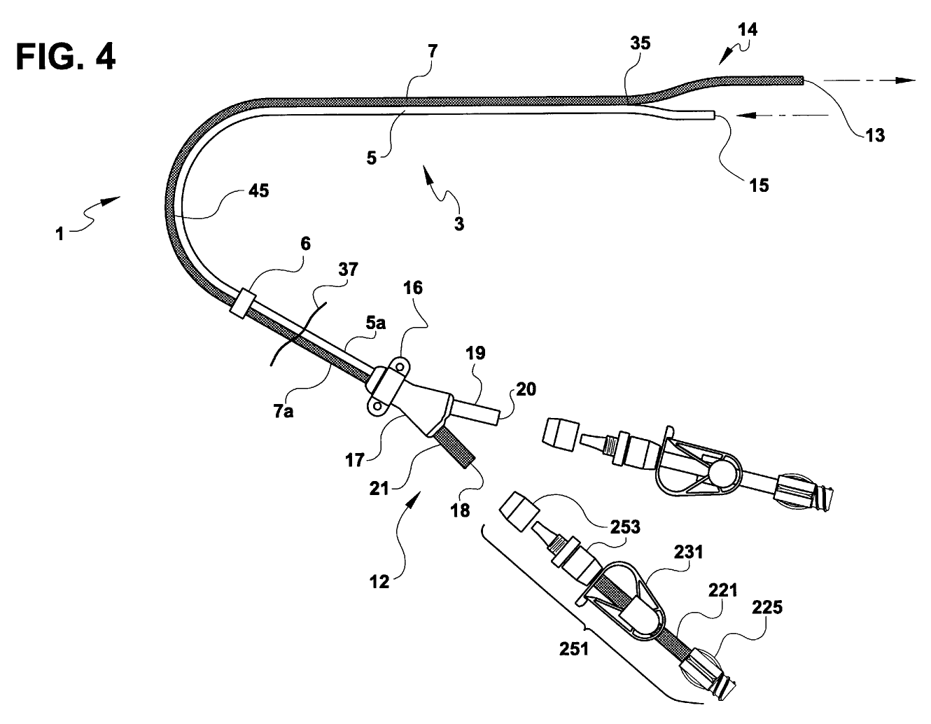

Referring to FIG. 4, catheter 3 is shown implanted through skin incision site 37 after the original extension clamps and luer connectors have been removed by cutting through extension tubes 19, 21 as part of the repair process, such as, for example, to remove and repair a malfunctioning extension tube section. After the extension tubes 19, 21 have been cut, the practitioner cannot readily differentiate the arterial tube 5 from the venous tube 7 on a conventional catheter. With the catheter disclosed herein, the extension tubes 19, 21 can be cut along lines 18 and 20. In one aspect, the venous tube 7 can be comprised of a different color than arterial tube 5, allowing for visual perception and identification by a user of at least a portion of each extension tube segment 5a and 7a extending from the incision site 37, after the extension tubes 19, 21 have been cut in preparation for a repair procedure.

In one aspect, at least one of extension tubes 19, 21 can be color coded to assist the user in visually identifying at least one of the arterial tube 5 or the venous tube 7. Using color coding as a guide to identifying each tube 5, 7, the user may repair the catheter 3 using a repair kit. In one aspect, the catheter repair kit can comprise a replacement assembly 251. In exemplary aspect, the replacement assembly 251 can comprise at least one replacement extension tube 221, at least one clamp 231, at least one compression fitting 253 or other sealing means for sealing or securing at least a portion of the replacement extension tube 221 to the catheter assembly 1, and at least one luer connector 225, as illustrated in FIG. 4. In one aspect, the compression fitting 253 is configured to effect a mechanical fluid-tight connection between the at least one extension tube of the catheter assembly 1 and the at least one replacement extension tube 221. In one aspect, the clamps 33, 31 can be releasably attached to at least a portion of each extension tube 19, 21, respectively. In one aspect, the clamps 31, 33 can be releasably attached to at least a portion of the outer surface of at least one of the extension tubes 19, 21. Optionally, the at least one replacement extension tube 221 can have an identical color to at least a portion of at least one of the extension tubes 19, 21 of the catheter assembly 1, as indicated by the identical shading of extension tube 21 and replacement tube extension section 221. Using at least a portion 5a, 7a of at least one of catheter tubes 5, 7 as a guide, a practitioner can match the color coded luers 225, replacement extension tube 221, and clamps 231 of the replacement assembly 251 with the similarly colored partial extension tubes 19, 21 of the implanted catheter assembly 1. In one aspect, the repair kit can be used to repair or lengthen at least one of the extension tubes 19, 21 of the catheter shaft 3.

The method of repairing a catheter assembly 1 described herein can involve cutting off at least a portion of an extension tube of a catheter assembly, providing a catheter assembly repair kit, as described herein, attaching at least a portion of the sealing means to at least a portion of the extension tube of the catheter assembly by matching at least one characteristic of the replacement extension tube with an identical characteristic of at least a portion of the extension tube of the catheter assembly, and securing at least a portion of the extension tube of the catheter assembly to at least a portion of the sealing means of the repair kit. In one exemplary aspect, the identical characteristic can be color. In one exemplary embodiment, at least a portion of one of the extension tubes of the catheter can have at least one identical physical characteristic as that of least a portion of at least one of the first tube and the second tubes 5, 7 of the catheter 1, as illustrated by the shading in FIGS. 1-5.

A method of repairing a catheter is also provided herein. This method involves providing a catheter having at least one extension tube; cutting at least a portion of at least one extension tube of the catheter; and providing a repair kit. The repair kit has at least one replacement extension tube, and at least a portion of the replacement extension tube is identical in at least one visible color to at least a portion of at least one extension tube of the catheter. The repair kit also has at least one means for sealing at least a portion of the replacement extension tube to at least a portion of the extension tube of the catheter. The method further involves attaching at least a portion of the sealing means to at least a portion of the extension tube of the catheter by matching the at least one visible color of the replacement extension tube with an identical visible color of at least a portion of the extension tube of the catheter and securing at least a portion of the extension tube of the catheter to at least a portion of the sealing means of the repair kit.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. The words “including” and “having,” as used herein including the claims, shall have the same meaning as the word “comprising.” Those familiar with the art can recognize other equivalents to the specific embodiments described herein, which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

Therefore, it is to be understood that the embodiments of the invention are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims.

This completes the description of the selected embodiments of the invention. Those skilled in the art can recognize other equivalents to the specific embodiments described herein which equivalents are intended to be encompassed by the claims attached hereto.

1. A multilumen venous access catheter, comprising:
a first catheter tube having at least one physical characteristic; and
a second catheter tube having at least one physical characteristic,
wherein the at least one physical characteristic of the respective first and second tubes are different.

2. The catheter of claim 1, wherein at least a portion of the first tube is joined to at least a portion of the second tube.

3. The catheter of claim 1, wherein the at least one physical characteristic corresponds to at least one visible color.

4. The catheter of claim 1, wherein at least a portion of the first tube differs in at least one visible color from at least a portion of the second tube, and wherein the at least one characteristic of each tube is identifiable according to the at least one visible color.

5. The catheter of claim 1, wherein the at least one physical characteristic of the first tube comprises at least one of hardness, tensile strength, rigidity, flexibility, radiopacity, torquability, trackability, pushability, fatigue strength, arterial tube function, venous tube function, abrasion resistance, and physical configuration.

6. The catheter of claim 1, wherein the at least one physical characteristic of the second tube comprises at least one of hardness, tensile strength, rigidity, flexibility, radiopacity, torquability, trackability, pushability, fatigue strength, arterial tube function, venous tube function, abrasion resistance, and physical configuration.

7. The catheter of claim 3, wherein the at least one physical characteristic is the cross-sectional lumen configuration.

8. The catheter of claim 1, wherein the first tube and the second tube further comprise a sidewall, and wherein at least a portion of the sidewall of the first tube is thicker than the second tube sidewall.

9. The catheter of claim 1, wherein the first tube further comprises a sidewall, and wherein the first tube sidewall has a variable thickness.

10. The catheter of claim 1, wherein the first tube comprises a first percentage by weight of radiopaque filler material, and the second tube comprises a second percentage by weight of radiopaque filler material, and wherein the second percentage by weight of radiopaque material is substantially greater than the first percentage by weight of radiopaque filler material.

11. The catheter of claim 1, wherein the first tube comprises a first durometer and the second tube comprises a second durometer, and wherein the first durometer is greater than the second durometer.

12. The catheter of claim 1, wherein the first tube has a first surface and the second tube has a second surface, and wherein at least a portion of the surface of the first tube and at least a portion of the surface of the second tube are joined together for at least a portion of the longitudinal length of the catheter.

13. The catheter of claim 1, wherein the first tube is an arterial tube and the second tube is a venous tube.

14. The catheter of claim 13, wherein the arterial tube comprises a lumen, and wherein the venous tube comprises a lumen, and wherein the arterial lumen cross-sectional area is different from the venous lumen cross-sectional area.

15. The catheter of claim 13, wherein the arterial tube is configured to prevent the collapse of the arterial tube during use.

16. The catheter of claim 13, wherein the venous tube is configured to optimize the functioning of the venous tube during use.

17. The catheter of claim 1, wherein the catheter further comprises at least one extension tube, and wherein at least a portion of the at least one extension tube comprises at least one identical physical characteristic as at least a portion of at least one of the first tube and the second tube.

18. The catheter of claim 1, wherein the catheter is a hemodialysis catheter.

19. A method for using a vascular access catheter, wherein the method comprises:
providing a catheter having an arterial tube and a venous tube, wherein the catheter comprises a means for identifying at least one physical characteristic of the arterial tube and a means for identifying at least one physical characteristic of the venous tube, wherein at least one physical characteristic of the respective arterial and venous tubes are different;
perceiving the means for identifying at least one characteristic of at least one of the arterial tube and the venous tube; and
identifying the at least one characteristic of at least one of the arterial tube and the venous tube in response to perceiving the means for identifying at least one characteristic of at least one of the arterial tube and the venous tube.

20. The method of claim 18, wherein the step of perceiving further comprises perceiving at least one visible color of at least one of the arterial tube and the venous tube, and wherein the at least one visible color corresponds to the at least one physical characteristic of at least one of the arterial tube and the venous tube.

21. The method of claim 18, wherein the step of identifying further comprises identifying the at least one characteristic of at least one of the arterial tube and the venous tube based on the at least one visible color.

22. The method of claim 18, wherein perceiving the at least one characteristic comprises perceiving at least one of the following: hardness, durometer, rigidity, flexibility, radiopacity, torquability, trackability, pushability, fatigue strength, abrasion resistance, arterial tube function, venous tube function, and physical configuration.

23. A method of repairing a catheter, wherein the method comprises:
providing a catheter having at least one extension tube;
cutting at least a portion of at least one extension tube of the catheter;
providing a repair kit, wherein the repair kit comprises at least one replacement extension tube, wherein at least a portion of the replacement extension tube is identical in at least one visible color to at least a portion of at least one extension tube of the catheter, and at least one means for sealing at least a portion of the replacement extension tube to at least a portion of the extension tube of the catheter;
attaching at least a portion of the sealing means to at least a portion of the extension tube of the catheter by matching the at least one visible color of the replacement extension tube with an identical visible color of at least a portion of the extension tube of the catheter; and
securing at least a portion of the extension tube of the catheter to at least a portion of the sealing means of the repair kit.
